(19) United States
(12) Patent Application Publication
(10) Pub. No.: US 2004/0054375 A1
(43) Pub. Date: Mar. 18, 2004

(54) Methods and apparatus for vessel ligation

(75) Inventors: Russell Aaron Houser, Livermore, CA (US); Lon Sutherland Annest, Tacoma, WA (US); Wendel Julio Smith, Tacoma, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Liganex LLC, Tacoma, WA

(21) Appl. No.: 10/412,558

(22) Filed: Apr. 11, 2003

Related U.S. Application Data
(60) Provisional application No. 60/411,496, filed on Sep. 18, 2002.
(60) Provisional application No. 60/424,585, filed on Nov. 6, 2002.
(60) Provisional application No. 60/426,550, filed on Nov. 15, 2002.
(60) Provisional application No. 60/428,501, filed on Nov. 22, 2002.

(51) Int. Cl. A61B 17/12

(52) U.S. Cl. 606/144

(57) ABSTRACT

Surgical fasteners comprise a needle coupled to a pair of closeable clips. The needle of the fastener may be penetrated through a blood vessel or other body lumen or duct which permits the clips to close around the exterior of the vessel to close the interior of the vessel.

### CROSS-REFERENCES TO RELATED APPLICATIONS

This application claims the benefit of prior provisional application No. 60/411,496 (Attorney Docket Nos. 21464-000100US), filed on Sep. 18, 2002; 60/424,585 (Attorney Docket No. 21464-000110US), filed on Nov. 6, 2002; 60/426,550 (Attorney Docket No. 21464-000120US), filed on Nov. 15, 2002, and 60/428,501 (Attorney Docket No. 21464-000130US), filed on Nov. 22, 2002. The full disclosures of each of these applications are incorporated herein by reference.

### BACKGROUND OF THE INVENTION

1. Field of the Invention

The present invention relates generally to medical methods and apparatus. In particular, the present invention relates to methods and apparatus for ligating body vessels, such as blood vessels.

Ligation is a surgical term referring to the binding or closure of a blood vessel or other body duct or lumen. Usually, suture, wire, or other ligature, is bound and constricted over the body vessel to close the lumen therethrough. Such ligation may be performed to isolate tissue from the vasculature, typically prior to organ removal or other tissue resection. Other purposes include fallopian tubes ligation for sterilization, and the like.

Lung resection surgery is a common treatment for lung cancer. Prior to resecting the lung tissue, the target region of lung must be isolated from the vasculature, typically requiring the ligation of one or two veins and three or more arteries. Common present techniques for blood vessel ligation include double ligation with suture for the use of staplers.

Shortcomings of existing methods include the need for large incisions where simple suture ligation is employed, the need for or a technically cumbersome process where minimally invasive suture tying techniques are used. Automatic devices, such as staplers, are expensive and are limited in the direction from which they can be fired. The anvils against which the staples fire are generally thick and require considerable dissection and more than ideal vessel length (from a tumor-margin standpoint). Simple suture ligation without through and through fixation carries the risk of rolling off the severed end of the ligated vessel and possible exsanguination in the post-surgical period.

For these reasons, it would be desirable to provide improved methods and apparatus for performing body vessel ligation. In particular, it would be desirable to provide relatively simple and inexpensive fasteners which permit the rapid and secure ligation of blood vessels and other body ducts and lumens. It would also be ideal if such devices could be deployed with one simple maneuver in a suture-placement-like technique familiar to surgeons. In the case of tissue removal for malignancy, a method that successfully achieves double ligature and division within shorter length of exposed/dissected vessel would provide improved margin (distance from malignant cells), and therefore possible improved cure rate. At least some of these objectives will be met by the inventions described below.

2. Description of the Background Art

Surgical clips incorporating needle structures are described in U.S. Pat. Nos. 6,503,260; 6,149,658; and 6,074,401; and U.S. Published Application Nos. 2002/0010490 and 2001/0018593. Corresponding PCT Publications include WO 02/087425; WO 02/030295; WO 01/174254; WO 01/017441; WO 00/059380; WO 99/062409; and WO 99/062406.

### BRIEF SUMMARY OF THE INVENTION

The present invention provides improved methods and devices for ligating blood vessels and other body lumens and ducts. In particular, the methods and devices of the present invention rely on penetrating at least one fastener through the target vessel or duct and subsequently closing the fastener over an exterior surface of the vessel or duct to close the lumen therein. Such methods and devices are particularly useful in performing lung resection procedures where multiple arteries and veins need to be ligated prior to removing the lung tissue. The methods and apparatus, however, are not limited to use in lung resection or with blood vessels, and may find use with a wide variety of other blood vessels, such as in variocele procedures, as well as with other body ducts, such as ligation of the fallopian tubes in sterilization procedures; a bronchus or other airway; a fistula or other cystic tract, any hollow viscous such as an appendicle stump, a bile or other excretory duct, or any other structure, vessel, duct, lumen, or other natural created body passageway to be permanently or temporarily interrupted.

According to a first aspect of the present invention, methods for ligating a tubular body vessel comprise penetrating one or more fastener(s) through opposed wall portions of the vessel. The fastener(s) are then closed over an exterior surface of the vessel wall, typically along a circumferential line, to close an interior lumen of the vessel. Often, at least two fasteners are penetrated through the opposed wall portions, typically through a common hole, where the fasteners are deformed over the vessel and opposed circumferential directions. Optionally, the two fasteners may be joined together while being penetrated. Alternatively, the two fasteners may be physically separate while being penetrated. In exemplary embodiments, the two fasteners are passed generally through the center of the target vessel and are closed in diametrically opposed directions to form a pair of C-clamps, each covering approximately one-half the vessel and disposed in an opposite direction.

In specific embodiments of the methods of the present invention, one or more additional fastener(s) or pairs of fasteners, may be penetrated through the body lumen at axially spaced-apart locations thereon. Typically, the fasteners will be identical to those introduced at the first location in the body vessel, and thus may be introduced using the same technique or introducer apparatus. Placement of multiple, axially spaced-apart fasteners can provide redundant closure in order promote the integrity of the seal. Additionally or alternatively, the vessel may severed or otherwise resected between such adjacently placed fasteners.

The fasteners will be suitable for penetration through the tubular body vessel and for subsequent closure over the exterior surface thereof. The fasteners may have a wide variety of geometries and may be formed from a wide variety of materials. Particular geometries include wires, coils, strips, rods, clips, twist ties, T-ties, ratcheting closures, interlocking closures, and the like. Particular materials include metals and polymers.

In a first preferred embodiment, the fasteners will be self-closing, typically being fabricated from an elastic material, a superelastic material, a shape-memory material, a heat-memory material, or combinations thereof. Exemplary elastic materials include spring stainless steels, such as Inconel, Monel, 17-7PH, and the like. Exemplary superelastic materials include superelastic metals, such as Nitinol, as well as superelastic polymers. In general, such self-closing structures will have an open configuration, typically maintained while the fastener is being delivered and/or while the fastener is being penetrated into the target body vessel, and a fully or partially closed configuration which is assumed after the fastener is penetrated into and positioned within the body vessel. Usually, the fastener will be constrained to be held in the open configuration during delivery and will be released from constraint to permit closing of the fastener over an exterior surface of the vessel to close the interior lumen thereof. Alternatively, such self-closing fasteners may be delivered in the closed configuration and temporarily opened during deployment to capture the exterior surface of the body vessel or some portion thereof. After capturing the vessel, the fastener may be released to close over the vessel in the desired manner.

In another preferred embodiment, the fasteners may comprise or be composed partly or wholly from a malleable or deformable material, typically metal, which is capable of non-elastic deformation. Such non-elastically deformable fasteners will be penetrated through the tubular body vessel in a straightened or otherwise opened configuration and then closed over the exterior surface of the vessel by applying force to “crimp” the fastener thereover. Exemplary malleable materials include stainless steel and the like.

In a third alternative construction, the fastener may comprise ratchets, detents, or other interlocking components to permit closure and securing of the fastener over the exterior of the tubular body vessel.

The fasteners will be configured to have dimensions selected to accommodate particular target tubular body vessels. The target body vessels will typically have a diameter in the range from 2 mm to 20 mm, usually from 4 mm to 15 mm. The fasteners will be configured to cover and compress at least a portion of the exterior circumference of such body lumens, typically covering at least half, often covering the entire circumferential difference, and sometimes covering the vessel more than one time. For linear elements (and those that may be linearized), the lengths of the fasteners will typically be in the range from 40 mm to 150 mm, usually from 50 mm to 100 mm, but can be any suitable length to accommodate a particular body lumen.

In a second aspect of the present invention, apparatus are provided for ligating or otherwise closing tubular body vessels. In a first embodiment of the apparatus, a surgical fastener comprises a needle having a proximal end and tissue-penetrating distal end. A pair of closable clips extend rigidly from the proximal end of the needle, and the needle may be penetrated through tissue to place the closable clips in the body vessel according to the methods described above. In a second embodiment, the surgical fastener is generally the same except that the needle is attached to the closable clips by a flexible connector, such as a wire or suture tether. The clips are separately connected to the connector. While these apparatus are particularly intended for performing tubular ligation according to the methods of the present invention, they may also find use in other ligating and non-ligating tissue closure and tissue approximation methods.

Needles useful in the apparatus of the present invention may take a wide variety of conventional and specialized forms. The needles may be composed of a wide variety of conventional needle materials, including stainless steels, superelastic alloys, shape memory alloys, and the like. The use of superelastic alloys, such as Nitinol, would allow the needle to be deflected without deformation, thus permitting the needles to be delivered in a constrained configuration and released into a deployment configuration. The use of shape or heat memory allows the needle to be maintained at room temperature, e.g. when packaged, in one configuration and assume another configuration when warmed by body contact, direct application of heat, or other methods. Thus, the needles may be straight, curved, or have other configurations, and such configurations may vary over time. The needle will typically have at least one sharpened tip or end to permit tissue penetration, and may have more than one such tissue-penetrating end. In certain embodiments, the needle may be formed together with the fastener, with an intermediate tether, or with other components of the apparatus.

The flexible connector will join the needle at one end to the fastener(s) at the other end. The flexible connector may be composed of a metal, metal alloy, suture material, polymers, biodegradable materials, absorbable materials, or combinations thereof. Exemplary metal alloys include stainless steels, Nitinols, and the like. Exemplary sutures include single filament sutures, multiple strand sutures, stretchable sutures, non-stretchable sutures, solid sutures, hollow sutures, and sutures having irregular cross-sectional geometries. The suture may be attached or bonded to the needle at one end and/or the fasteners at the other end by any conventional technique, including swaging, crimping, sonic welding, soldering, heat forming, adhesives, solvent bonding, and combinations thereof. The material of the flexible connector may be bonded to the needle and/or fastener externally, internally, or by any combination thereof, either at the ends or at locations based inwardly from the ends of each element.

The term fastener is intended to cover any type of clip, ligating device, or other structure which is capable of ligating tubular body vessels as described herein. In particular, the terms fasteners, clips, ligating devices, and ligating elements will be used interchangeably in this text. The fasteners may be wires, coils, strips, rods, combinations thereof, or have any other suitable cross-sectional geometry. At least one surface of the fastener in the resting or deployed configuration may be flat, concave, convex, round, coiled, criss-crossed (FIG. 8), or have other configurations. The fasteners may have an interlocking geometry to enable or assist with compression of the tissue. The fasteners may be self-closing (compressing), typically being fabricated from an elastic or superelastic material. The use of superelastic fasteners would allow significant deflection or deformation of the fasteners while being delivered without causing any permanent deformation. Such superelastic properties also facilitate delivery configurations where the device is delivered in one configuration and released or induced to change into another configuration. The fasteners may have a wide variety of geometries suitable for compressing vessels of different sizes. For example, the fasteners may be long enough to compress an entire section of a vessel, or half the section of the vessel, or may be long enough to wrap entirely around the vessel one or more times. In the latter case, the use of longer fasteners would result in further wrapping of the outside of the vessel, facilitating the selection and sizing of the fasteners to be used.

In certain embodiments, the fasteners may be separated from the flexible connector and/or the needle by cutting, cleating, or any other method or process. In some embodiments, the fasteners may be detached from the flexible connector simply by bending the two elements to an acute angle. In other embodiments, the transition region between the fastener and the flexible connector and/or needle may include a frangible section, such as a notch, hole, cut-out, groove, reduced cross-section, or otherwise weakened area, to permit the rapid detachment of the fastener by bending or other action. The fasteners may be treated in a variety of conventional or unconventional ways such as coating, jacketing, overmolding, dipping, spraying, casting, or combinations thereof. Such layers, coatings, or other materials may be intended to provide a softer contact area, provide a drug elution layer, or the like.

In the first embodiment of the surgical fastener, a frangible segment is disposed between the needle and the clips to allow selective detachment of the needle after the clips have been implanted in tissue. Typically, although not necessarily, the frangible segment will be disposed at a transition location between the needle and the closable clips. In a preferred embodiment, the needle and clips will comprise a single, continuous structure, although it will also be possible to form the needle and clips separately and provide for a rigid joint between such components.

The clips will be closable over tissue by any of the techniques described above in connection with the methods of the present invention. In particular, the clips may have an elastic memory and be formed into a closed configuration. The clips may then be opened prior to or during deployment and allowed to close over the tissue after deployment. The clips may also be formed from a malleable or otherwise non-elastically deformable material. Such malleable clips may be deployed in a generally open configuration, and be subsequently closed by applying a force to the clips after they have been properly located in the tissue. In either case, after deployment, the needle may be detached from the clips by breaking the structure at the frangible point (if provided).

In the second embodiment of the surgical fastener, the flexible connector will usually be bifurcated with one closeable clip attached to each bifurcation. Advantages of this configuration include the through and through fixation of the clip with circumferential application of the device while requiring only a single pass of the penetrating member.

In both embodiments of the apparatus, the flexible connectors may comprise or be composed of any one of a wide variety of materials, including suture, elastic metal, superelastic metal, elastically deformable metals, non-elastically deformable metals, biodegradable polymers, bioabsorbable polymers, non-degradable polymers, and the like. The closable clips of the apparatus may be configured as described in connection with the methods as described above.

### BRIEF DESCRIPTION OF THE DRAWINGS

FIGS. 1A through 1C are schematic illustrations of the method of the present invention for ligating a tubular body lumen.

FIG. 2 illustrates a first exemplary embodiment of an apparatus useful for performing the methods of the present invention, including a pair of closable clips.

FIGS. 3A through 3B illustrate exemplary closable clips useful with the apparatus of FIG. 2.

FIGS. 4A through 4E illustrate a method according to the present invention for ligating blood vessels extending from the pulmonary artery to the lung in accordance with the present invention.

FIGS. 5A and 5B illustrate an alternative embodiment of an apparatus according to the present invention.

FIGS. 6A through 6C illustrate the use of the device of FIGS. 5A and 5B for ligating a tubular body vessel in accordance with the principles of the present invention.

FIGS. 7A and 7B illustrate a third alternative fastener apparatus constructed in accordance the principles of the present invention.

FIGS. 8A and 8B illustrate a fourth exemplary tissue-fastening apparatus constructed in accordance with the principles of the present invention.

FIGS. 9A and 9B illustrate a fifth alternative embodiment of a tissue fastener constructed in accordance with the principles of the present invention.

FIGS. 10A and 10B are views of a clip applier according to the present invention.

FIGS. 11A through 11D illustrate use of the clip applier of FIGS. 10A and 10B in ligating a vessel according to the methods of the present invention.

### DETAILED DESCRIPTION OF THE INVENTION

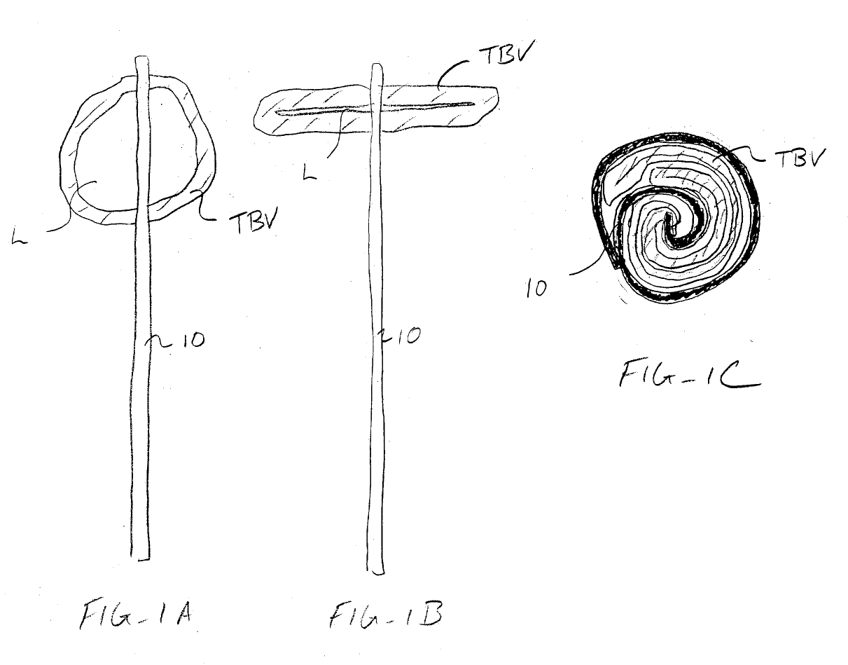

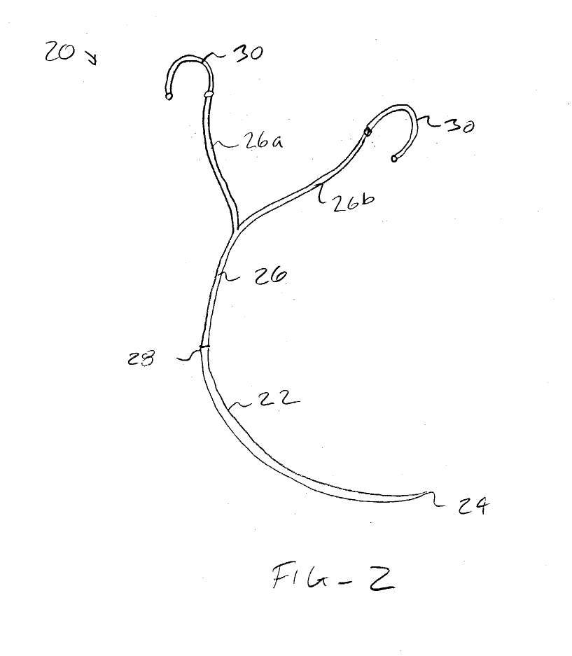

Referring now to FIGS. 1A through 1C, methods according to the present invention for ligating a tubular body vessel TBV are described. Initially, a fastener 10 is penetrated through the tubular body vessel TBV so that it passes through opposed wall portions, as illustrated in FIG. 1A. The opposed wall portions are preferably collapsed toward each other, as illustrated in FIG. 1B, and the fastener 10 then closed over the exterior surface of the vessel wall to close the interior lumen L of the vessel, as shown in FIG. 1C. In particular, the fastener 10 is shown to form a spiral coil which captures and traps the flattened wall portions of the vessel. Such coiling may be achieved in any of the ways discussed in the Summary above. In particular, the fastener may be composed of an elastic material having a spirally-coiled “memory.” The fastener may then be straightened, e.g., by placement in a linear sheath (not shown), and introduced through the walls of the target blood vessel TBV while in said straightened configuration. By then removing the sheath or other constraint, the fastener 10 will resume its coiled configuration, thus entrapping the tissue as shown in FIG. 1C. Alternatively, the fastener may be formed from a non-elastically deformable material. In the latter case, the fastener may then be coiled over the tissue by applying a force to impart elastic deformation.

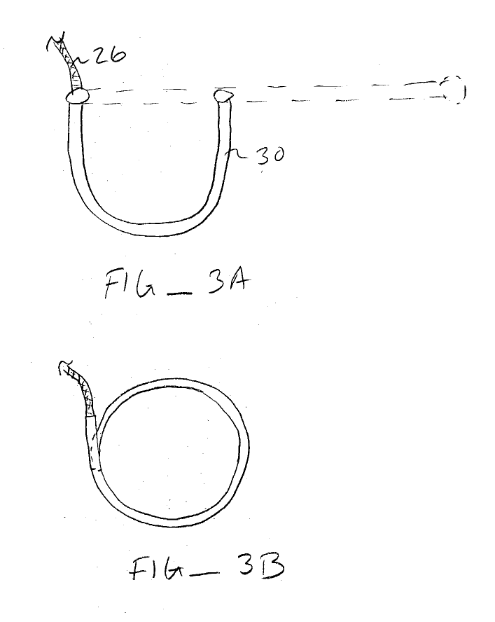

Referring now to FIG. 2, a surgical fastener assembly 20 constructed in accordance with the principles of the present invention comprises a needle 22 having a tissue-penetrating distal end 24, typically a sharpened distal tip. A flexible connector 26 is attached to a proximal end 28 of the needle 22 and is connected at its opposite end to a pair of separate closable clips 30. As illustrated, the flexible connector is bifurcated and includes separate legs 26a and 26b. The closable clips 30 are attached separately to each of the separate legs. This bifurcated design is desirable since two or more smaller radius clips can apply more occluding force than one larger radius clip (LaPlace's Law). Also, they simultaneously allow ligation circumferentially and through-and-through fixation.

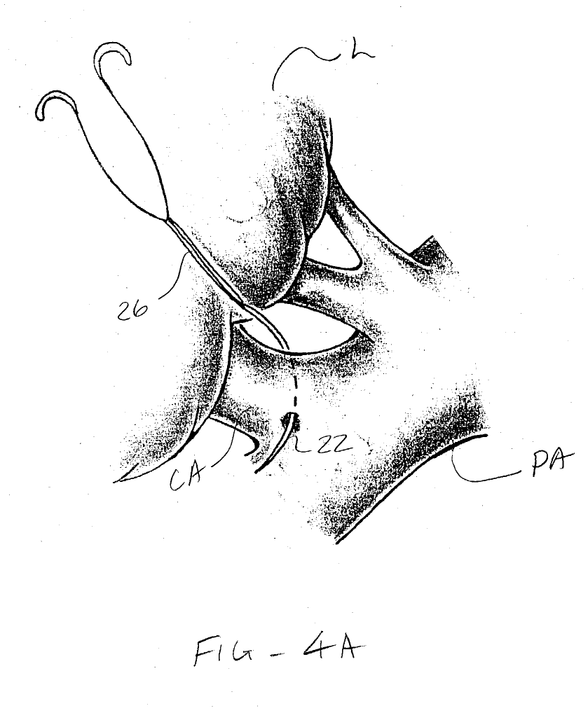

The clips 30 (FIG. 3A) have a generally U-shaped configuration and are preferably formed from an elastic material, usually superelastic or shape-memory material, so that they may be elastically deformed between a straightened configuration (shown in broken line) and the U-shaped configuration (full line) of FIG. 3A. In this way, the clips may be introduced into tissue while straightened and released from constraint to assume their tissue-closing configuration after they are properly located in tissue. Superelastic, shape-memory clips and other components may be processed as known to those skilled in the art, typically by constraining the component in a desired configuration, annealing at temperatures typically ranging from 400° C. to 600° C., for typically between 30 seconds and 20 minutes, quenching with ice water (or other suitable method) and repeating as desired to impart a desired resting configuration.

The closable clips may have a variety of other configurations. As illustrated in FIG. 3B, the clip may assume a fully circular, closed configuration when unconstrained. A configuration consisting of a single coil when “released” forms a spiraling ligature over the structure to be occluded. While the piercing member of the coil in this embodiment is passed through-and-through the structure, occlusion is accomplished by coiling of the elastic member around the outer circumference one or more times. The number of times the spiral wraps around the structure depends on the length of the coil relative to the size of the structure. The single coil configuration may be deployed through an injector-like device.

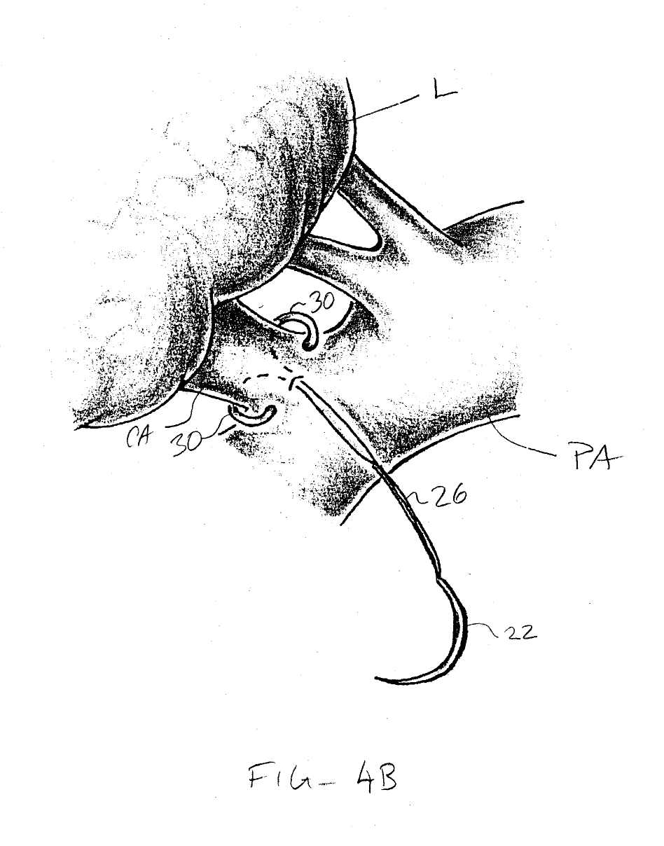

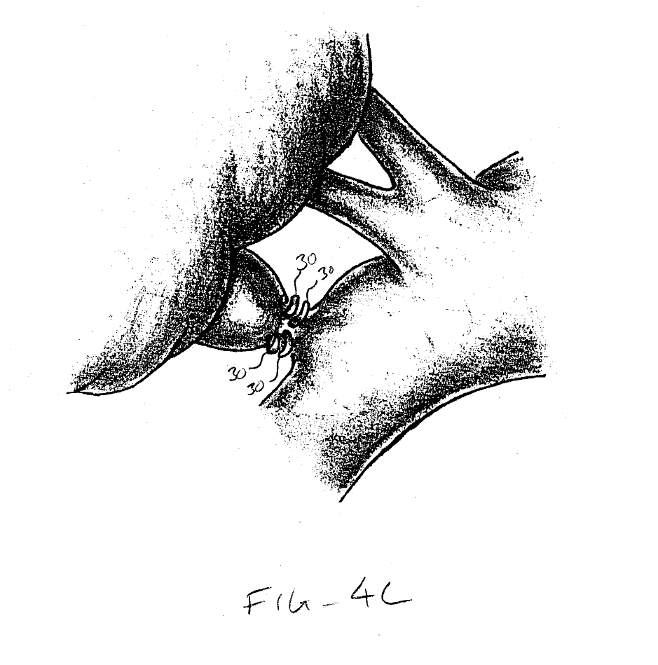

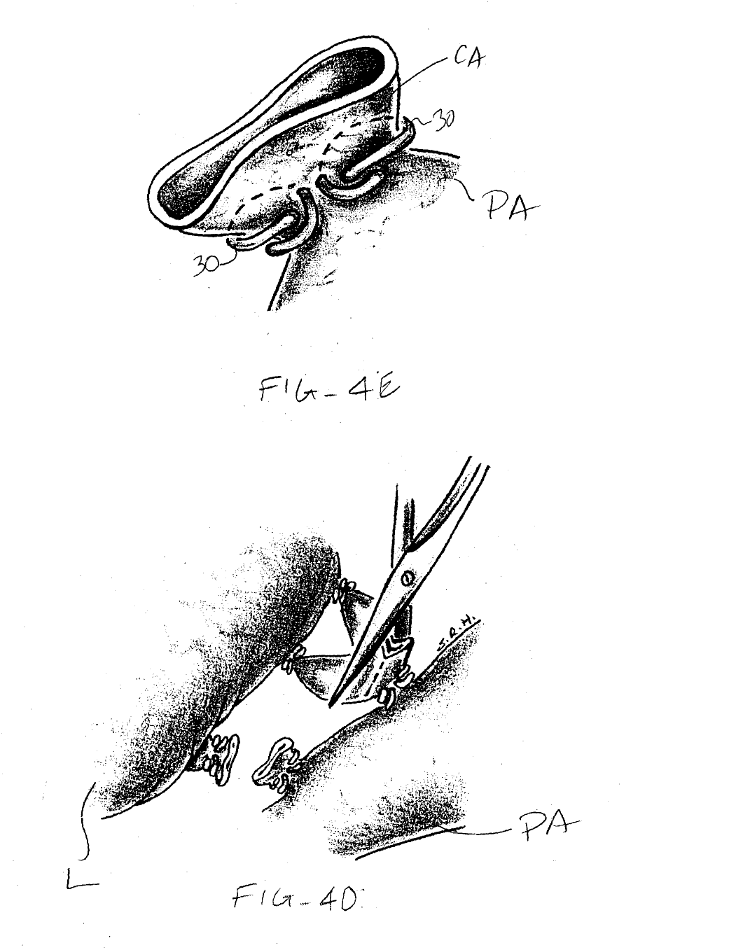

Referring now to FIGS. 4A through 4E, use of the assembly 20 for ligating a connecting artery CA between the pulmonary artery PA and lung L of a patient will be described. Initially, (FIG. 4A), the needle 22 is passed through the walls of the connecting artery CA near its base on the pulmonary artery PA. The needle is advanced to draw the flexible connector 26 through the needle penetration to draw the closable clips 30 over opposite sides of the connecting artery CA, as shown in FIG. 4B. The clips may then be crimped on to the opposite sides of the exterior of the connecting artery CA, and usually a second pair of clips placed in an axially spaced-apart location, as shown in FIG. 4C. Additional clips may then be placed at the upper portion of the connecting artery CA adjacent to the lung L. The connecting artery eventually is severed, as shown in FIG. 4D. A detailed view of a severed artery having a pair of clips 30 present to close the vessel lumen is shown in FIG. 4E.

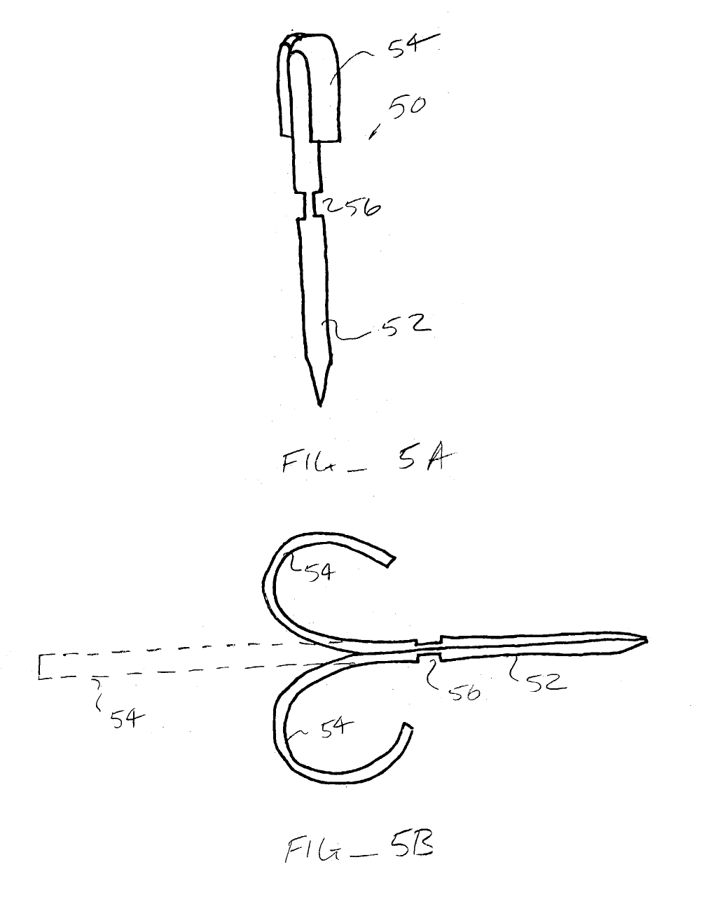

An alternative embodiment of a surgical fastener constructed in accordance with the principles of the present invention is illustrated in FIGS. 5A and 5B. Surgical fastener 50 includes a needle portion 52 secured to a pair of closable clips 54, where the clips are rigidly attached to the needle. Preferably, a frangible region 56 is disposed between the needle 52 and the clips 54.

The closable clips 54 are shown in their closed configuration and full line in FIGS. 5A and 5B. The clips are shown in a straightened, deployment configuration in broken line in FIG. 5B. The clips may be formed from an elastic material, in which case the closed configuration would be their shelf or unstressed configuration and the straightened configuration would require constraint or other force in order to be maintained. Alternatively, the clips may be formed of a malleable material in which case they could be formed and reformed between the straightened and closed configurations as illustrated.

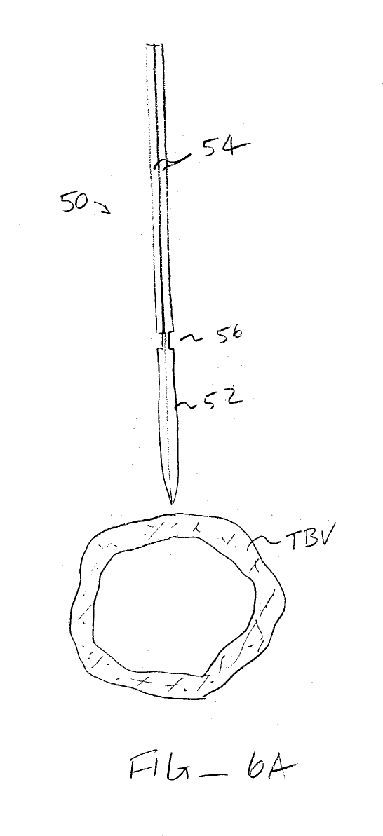

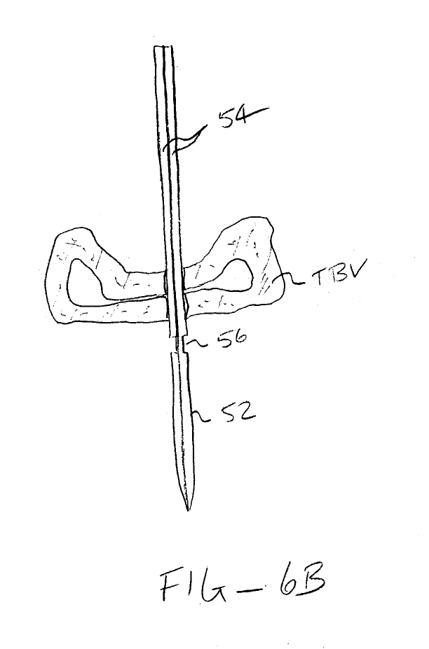

Use of the fastener 50 for ligating tubular vessel TBV is illustrated in FIGS. 6A through 6C. The fastener is penetrated into the body vessel to collapse the opposed walls thereof, as shown in FIGS. 6A and 6B. Preferably, the frangible section 56 will be placed just beyond the penetration through the lower (as illustrated in FIG. 6B) wall of the vessel. The needle 52 may then be broken off and the clips closed over the walls of the tubular body vessel, as shown in FIG. 6C.

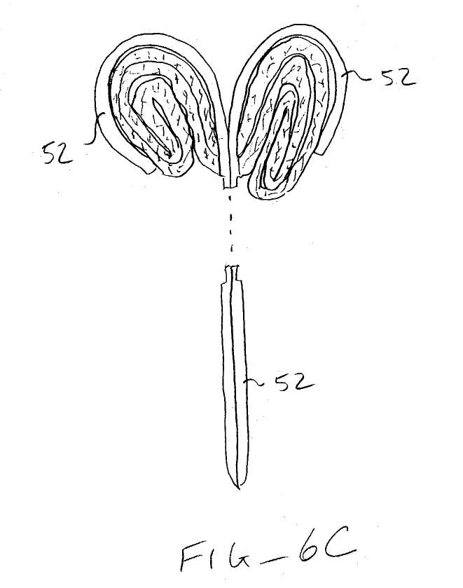

The fastener design of FIGS. 5A and 5B can be varied in a number of ways. For example, as shown in FIGS. 7A and 7B, a fastener 60 includes a needle portion 62 secured to a pair of closable clips 64. The needle portion 62 is rigidly secured to the clips 64, and a frangible region 66 is positioned therebetween. Fastener 60 differs from fastener 50 principally in the design of the clips 64 which include fork-like ends 68 which are configured to close so that the individual tines 70 of each fork 68 interdigitate when the clips 64 are closed about the exterior of a tubular body vessel BV, as shown in FIG. 7B.

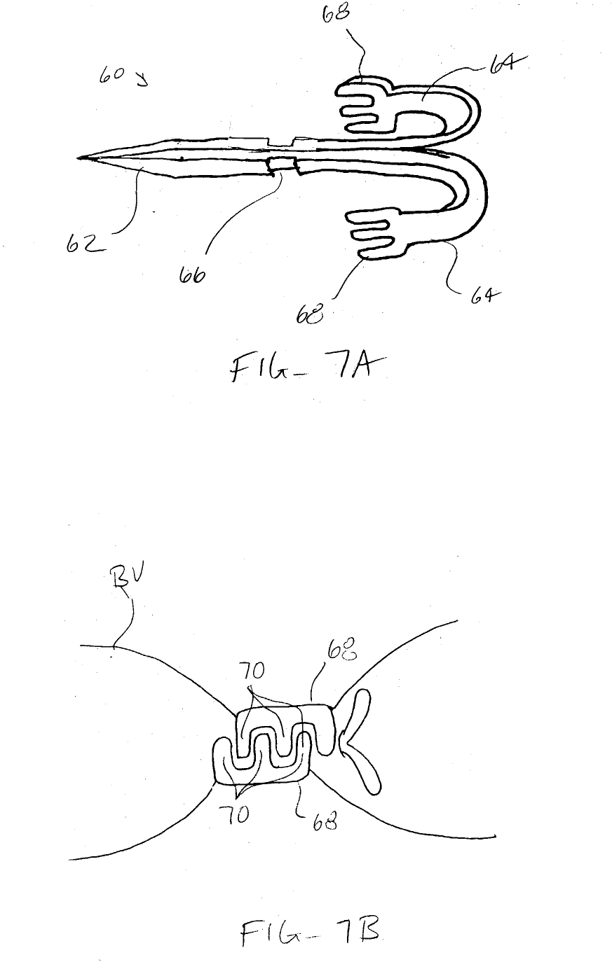

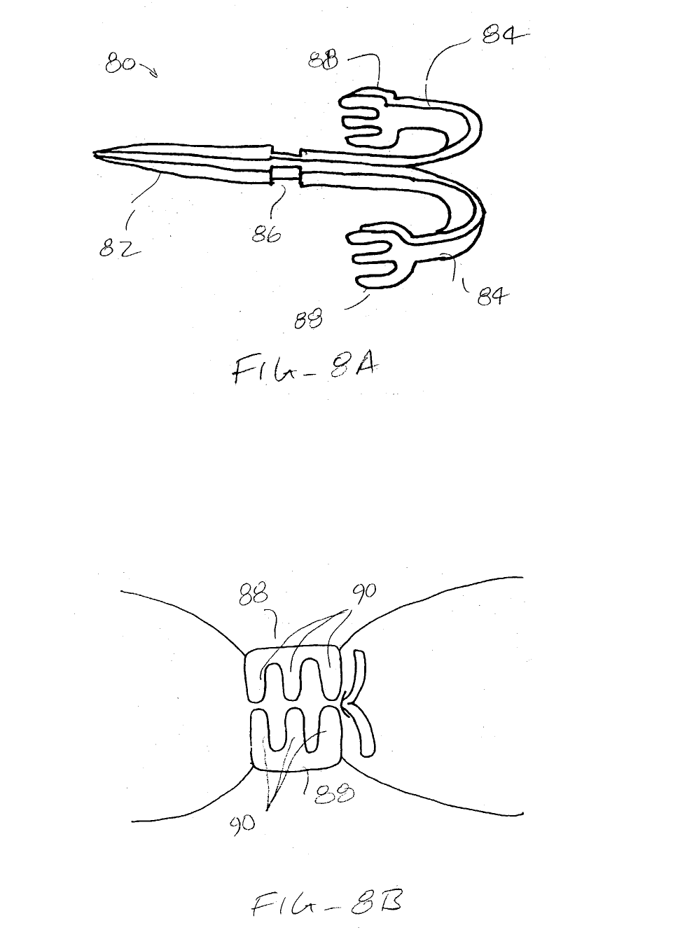

Fastener 80, as shown in FIGS. 8A and 8B, is very similar to fastener 60, including a needle portion 82, a pair of clips 84, a frangible region 86, and fork-like ends 88 on each of the clips 84. The principal difference between fastener 60 and fastener 80 is that fastener 80 is adapted so that the fork-like ends 88 close with the individual tines 90 aligned with each other, as shown in FIG. 8B, rather than in interdigitating as with fastener 60.

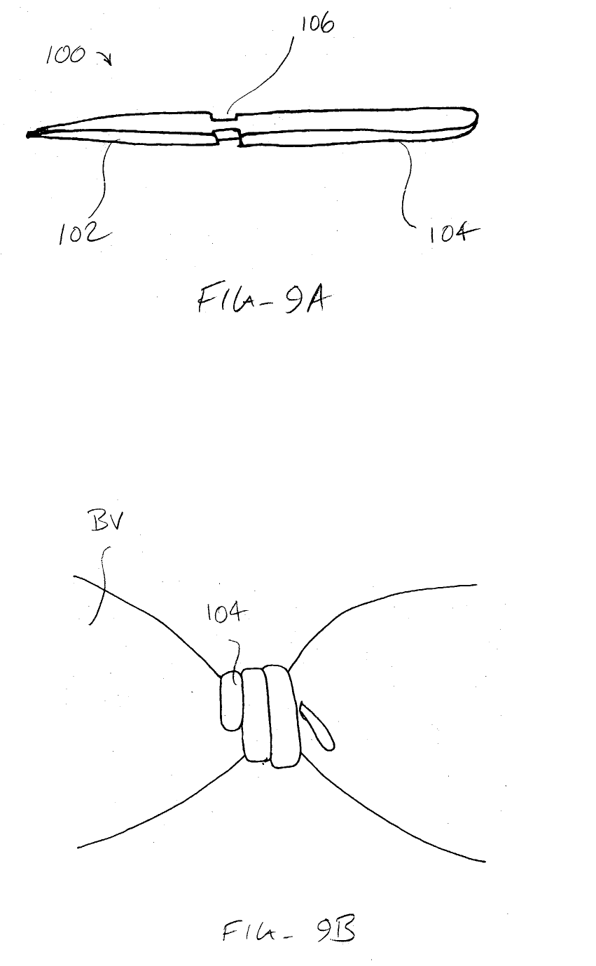

An additional fastener 100 is illustrated in FIGS. 9A and 9B. Fastener 100 also includes a needle portion 102, a clip portion 104, and a frangible region 106 joining the needle and clip portions. The clip portion 104 of fastener 100 may be malleable or elastic, usually being elastic so that it may be delivered in a straightened, constrained configuration and allowed to coil about the body vessel BV when released, as shown in FIG. 9B.

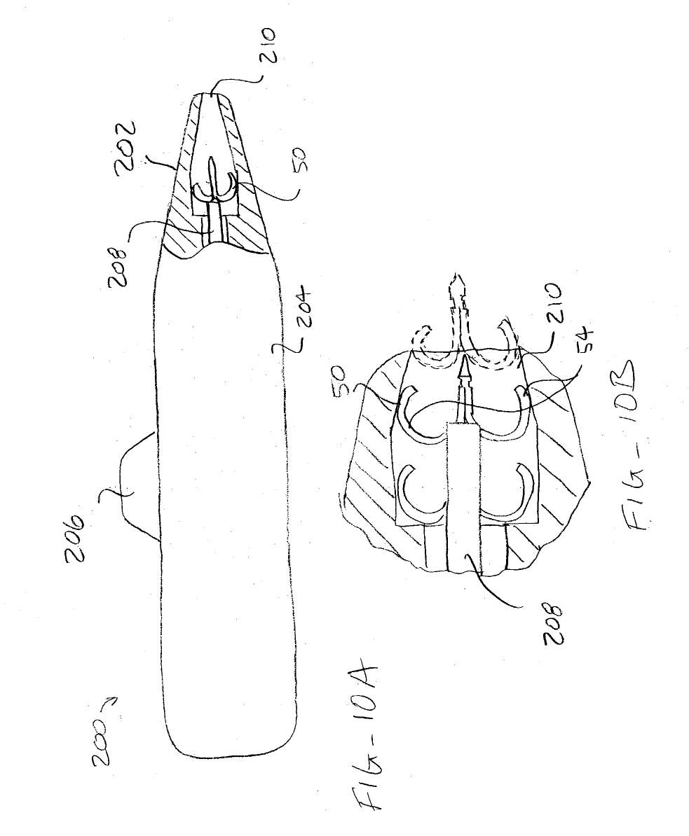

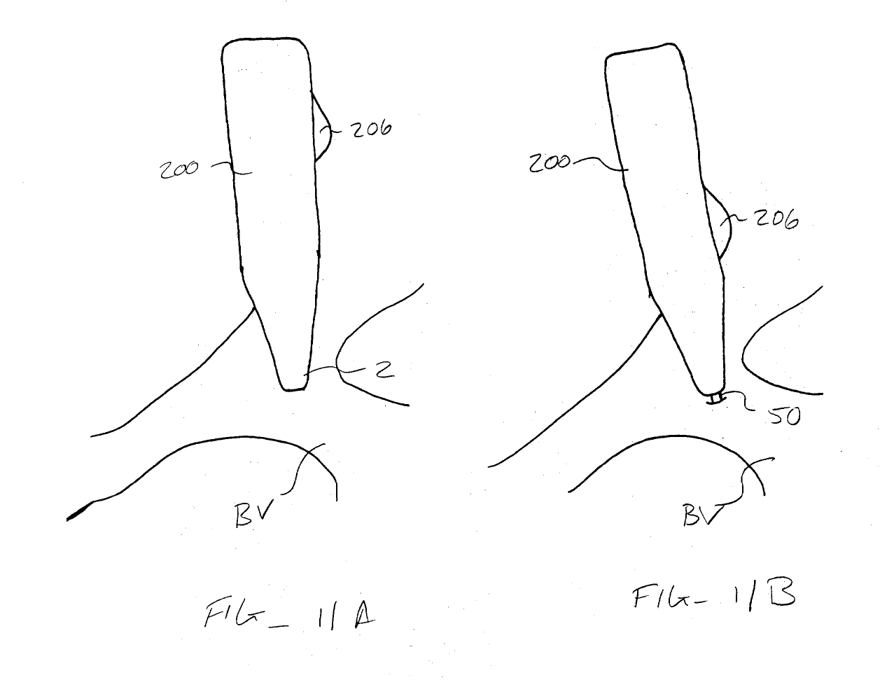

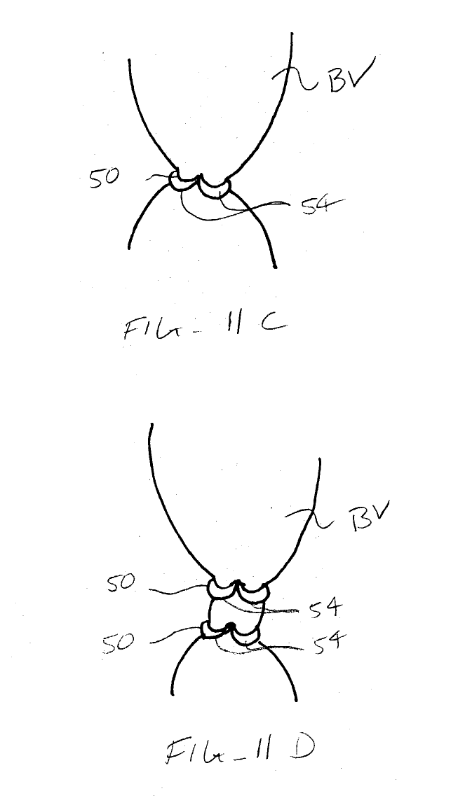

Referring now to FIGS. 10A and 10B, a fastener applier 200 is adapted for delivering a plurality of fasteners 50 from a magazine held in the distal end 202 of the applier. The applier 200 comprises a hand-held body 204 having a thumb slide 206 which can axially advance a pusher rod 208. The pusher rod 208, as shown in detail in FIG. 10B, engages individual fasteners 50 and advances them distally out an opening 210 at the distal tip of the applier. The magazine consists of a lateral stack of clips 50 with a spring or other apparatus (not shown) for individually advancing them to the pusher rod 208. As the fasteners 50 are distally advanced by the push rod, the clips 54 are laterally compressed as the fastener is deployed from the distal end 202. By holding the distal end 202 against tissue to be clipped or fastened, fastener 50 will directly enter into the tissue and be deployed, as desired in more detail with reference to FIGS. 11A-11D.

Referring now to FIGS. 11A-11D, the distal end 202 of the clip applier 200 is engaged against the location in the body vessel duct BV where it is desired to implant a clip. The thumb slide 206 is then manually advanced to deploy a first fastener 50 into the body vessel BV, as shown in FIG. 11B. The fastener 50 is deployed so that the individual clips 54 circumscribe and close the exterior of the body vessel BV, as shown in FIG. 11C. Usually, at least a second clip 50 will be applied at a spaced-apart location from the first clip, as shown in FIG. 11D. In this way, the body vessel duct BV can be severed between the two clips (not shown).

While particular embodiments of surgical fasteners useful for performing the methods of the present invention have been illustrated, it will be appreciated that a wide variety of other specific embodiments will be available.

What is claimed is:

1. A method for ligating a tabular body vessel, said method comprising:
penetrating one or more fastener(s) through opposed wall portions of the tubular body vessel; and
closing the fastener(s) over an exterior surface of the vessel wall to close an interior lumen of the vessel.

2. A method as in claim 1, wherein at least two fasteners are penetrated through the opposed wall portions.

3. A method as in claim 2, wherein he at least two fasteners are penetrated through a common hold and deformed in opposed circumferential directions.

4. A method as in claim 3, wherein the at least two fasteners are joined together while being penetrated.

5. A method as in claim 3, wherein the at least two fasteners are physically separate while being penetrated.

6. A method as in claim 2, wherein the at least two fasteners are closed to form diametrically opposed C-clamps.

7. A method as in claim 1, further comprising penetrating one or more additional fastener(s) through opposed wall portions which are axially spaced-apart along the tubular body vessel.

8. A method as in claim 1, wherein the fastener(s) comprise(s) an elastic material, and deforming comprises releasing the fastener from constraint to assume a memory configuration which constricts over the exterior surface.

9. A method as in claim 1, wherein the fastener(s) comprise(s) a non-elastically deformable material, and deforming comprises applying a shaping force to the fastener above the elastic limit of the material.

10. A method as in claim 1, further comprising severing the body lumen at a location adjacent to fasteners which have been closed.

11. A method as in claim 1, wherein the tubular body vessel is selected from the group consisting of a blood vessel; a fallopian tube; a bronchus; a fistula; a cystic tract; and appendiced stump; and a bile duct.

12. A surgical fastener comprising:
a needle having a proximal end and a tissue-penetrating distal end; and
at least one closable clip rigidly extending from the proximal end of the needle.

13. A surgical fastener as in claim 12, wherein a frangible segment is disposed between the needle and the clip to allow detachment of the needle after the clips have been implanted in the tissue.

14. A surgical fastener as in claim 12, wherein the needle and clip comprise a single, continuous structure.

15. A surgical fastener as in claim 12, wherein the needle and clips comprise separate structures which are joined at a rigid junction.

16. A surgical fastener as in claim 12, wherein the clips have an elastic memory and are formed into a closed configuration, whereby the clips may be opened during deployment and allowed to close after deployment.

17. A surgical fastener as in claim 12, wherein the clips are malleable, whereby the clips may be employed in a generally open configuration and a force applied to non-elastically close the clips after they have been deployed.

18. A surgical fastener assembly comprising:
a needle having a proximal end and a tissue-penetrating distal end;
a flexible connector extending from the proximal end of the needle; and
a pair of separate closable clips attached to the flexible connector.

19. A surgical fastener assembly as in claim 18, wherein the flexible connector is bifurcated with one closable clip attached to each bifurcation.

20. A surgical fastener assembly as in claim 18, wherein the flexible connector comprises a material selected from the group consisting of suture, elastic metal, superelastic metal, elastically deformable metal, biodegradable polymers, bioabsorbable polymers, and non-degradable polymers.

21. A surgical fastener assembly as in claim 12, wherein the clips have an elastic memory and are formed into a closed configuration, whereby the clips may be opened during deployment and allowed to close after deployment.

22. A surgical fastener assembly as in claim 12, wherein the clips are malleable, whereby the clips may be deployed in a generally open configuration and a force applied to non-elastically close the clips after they have been deployed.
